(19) United States
(12) Patent Application Publication
(10) Pub. No.: US 2002/0090655 A1
(43) Pub. Date: Jul. 11, 2002

(54) Method of purification of neuropeptide containing vesicles from the brain

(75) Inventors: Richard E. Fine, Chestnut Hill, MA (US); Eric A. Berg, Cumberland Center, ME (US)

Correspondence Address: DARBY & DARBY P.C., 805 Third Avenue, New York, NY, 10022, US

(73) Assignee: Trustees of Boston University

(21) Appl. No.: 10/001,898

(22) Filed: Oct. 31, 2001

Related U.S. Application Data
(60) Provisional application No. 60/244,971, filed on Nov. 1, 2000.

(51) Int. Cl. G01N 33/53; G01N 33/567; C12N 1/00

(52) U.S. Cl. 435/7.2; 435/317.1

(57) ABSTRACT

The invention is directed to a new method of isolating and purifying a large quantity of neuropeptide containing secretory vesicles (dense core vesicles) from a specific brain region and subsequently isolating and identifying novel neuropeptides contained therein. The method of the invention has significant practical value. The availability of large quantities of such vesicles will enable the identification of novel peptides using highly sensitive techniques, e.g., mass spectroscopy. Such neuropeptides are highly likely to have novel biological effects.

This invention was made with Government support under Contract Number R37AG-05984 awarded by the National Institutes of Health. The Government has certain rights in the invention.

### FIELD OF THE INVENTION

The present invention relates generally to the purification of neuropeptide containing vesicles from the central nervous system. In particular, the invention is directed to the large scale isolation of dense core vesicles.

### BACKGROUND OF THE INVENTION

The secretion of neurotransmitters, including neuropeptides, in the central nervous system (CNS) has been the focus of many researchers, and significant progress has been made in the delineation of exo/endocytotic pathways of synaptic secretory organelles (Palfrey and Artalejo, Neuroscience 1998, 83: 969-89; Kelly, Cell 1993, 72 Supp.: 43-53). The synaptic vesicle is the major secretory organelle in the synapse and contains both specific neurotransmitters and proteins with critical roles in neurotransmission (Sollner and Rothman, Trends Neuroscience 1994, 17: 344-8). There are subclasses of synaptic vesicles including classic synaptic vesicles including classic synaptic vesicles or clear synaptic vesicles and dense core vesicles. Classic synaptic vesicles (SV) contain neurotransmitters such as glutamate, acetylcholine, glycine, γ-aminobutyric acid, and iogenic amines while dense core vesicles (DCVs) contain neuropeptides, and in some cases catecholamines, as well as biogenic amines (Kelly, Cell 1993, 72 Supp.: 43-53; Bruns and Jahn, Adv. Pharmacol. 1998, 42: 87-90; Burger et al., Neuron 1991, 7: 287-93).

To date, most research involving DCVs and their components have used either cell lines (i.e., PC-12), isolated primary neurosecretory (chromaffin) cells, or vasopressin and oxytocin-containing hypothalamic magnocellular neurons (Elhamdani et al., J. Neuroscience 1999, 19: 7375-83; Klenchin et al., Methods 1998, 16: 204-8; Laslop, et al., Neuroscience 1994, 59: 477-85; Walch-Solimena et al., J. Neuroscience 1993, 13: 3895-903; Schmidle et al., Biochim Biophys Acta 1991, 1060: 251-6; Whitnall and Gainer, Brain Res. 1985, 361: 400-4). However, there remains in the art a need for a system of isolating and purifying neuropeptide containing vesicles, thereby producing a readily accessible research substrate. Further advantages are set forth more fully in the accompanying Description and Drawings.

The dense core vesicle, a subclass of synaptic vesicles, contains neuropeptides, and in some cases catecholamines and biogenic amines. Until recently, there had been little research on DCVs in neurons from the CNS. There is experimental evidence that proteins associated with classic SVs are present in DCVs. Researchers have demonstrated that the SV protein, synaptotagmin, is a membrane constituent of neuropeptide-containing large DCVs isolated from neurosecretory cell lines (PC-12) (Lah and Burry, J. Neurochem 1993, 60: 503-12; Cutler and Cramer, J Cell Biol 1990, 110: 721-30) as well as the hypothalamus and posterior pituitary (Walch-Solimena et al., J. Neurosci 1993, 13: 3895-903). However, there is conflicting data as to whether synaptophysin, a classic SV protein, is present in significant amounts in DCVs (Morin et al., Ann NY Acad Sci 1991, 632: 442-3; Winkler, Neurochem Res 1997, 22: 921-32; Bauerfeind et al., Proc. Natl. Acad. Aci. USA 1995, 92: 7342-6). Synaptobrevin has also been shown to be in DCVs (Papini et al., J. Biol. Chem. 1995, 92: 7342-6; Chilcote et al., J. Cell Biol. 1995, 129: 219-31) suggesting a similar mechanism of exocytosis to that of classic SVs.

DCVs contain granins and other secretory markers, which classic SVs do not (Fischer-Colbrie et al., Prog. Neurobiol. 1995, 46: 49-70; Winkler, J. Anat. 1993, 183: 237-52; Weiler et al., FEBS Lett. 1990, 265: 27-9). DCVs from noradrenergic neurons (De Potter et al., Synapse 1997, 25: 44-55) and the corpus striatum (Llona et al., Neurochem Int. 1994, 25: 573-81) contain both neuropeptides and catecholamines. Recently, experiments have shown co-localization of specific ion channels and DCV markers in cultured cortical (Chuang et al., J. Neurosci 1999, 19: 2919-28) and sympathetic (Paquet et al., J. Neurosci. 1996, 16: 964-73) neurons, as well as the identification of specific proteins in DCVs from the bovine splenic nerve (Leitner et al., J. Neurochem. 1999, 72: 1110-6).

As used herein, DCVs are the preferred embodiment of the focus on peptide-containing vesicles.

### SUMMARY OF THE INVENTION

The present invention provides a method of analyzing contents of dense core vesicles, from brain samples, which have been purified on a large scale. In one embodiment, the brain sample used is rabbit optic nerve. Analysis is carried out preferably by immunoassay Western blot, chromatography, mass spectroscopy, or immunoadsorbtion.

The present invention further provides a method of purifying dense core vesicles. This method includes the centrifugation of a resuspended pellet after homogenization of nerve and termini from dissected brain samples in order to obtain a microsome preparation. In a preferred embodiment, the centrifugation is done at a low speed followed by 100,000 g for 2 hours. The microsome preparation is then separated by a sucrose velocity size gradient and is centrifuged onto a sucrose pad to yield a purified product. The preferred embodiment would centrifugate at 92,000 for 18 hours. The preferred embodiment would further use the sucrose velocity size gradient at gradient levels between 29-42%. The large scale quantity of the purified product is then collected from an equilibrium density gradient. The preferred embodiment would use a sucrose equilibrium density at gradient levels between 38.5-45.5%.

The third aspect of thd invention provides a purified product of dense core vesicles containing the peptide of interest. In one embodiment, Substance P is the peptide marker with a purification greater than 1,750-fold.

### DESCRIPTION OF THE DRAWINGS

The foregoing and other features of the invention will be more readily appreciated from the following description of an exemplary embodiment taken in conjunction with the accompanying drawings, wherein:

FIGS. 1A and 1B disclose the separation of substance P-containing vesicles in a sucrose velocity gradient. Preparations of microsomes from ON and LGN/SC respectively, were centrifuged in a 10-50% sucrose velocity gradient and analyzed for substance P by RIA. A, Substance P distribution of fractionated ON microsomes; and B, Substance P distribution of fractionated LGN/SC microsomes by radioimmunoassay.

FIGS. 2A-2E disclose the fractionation of substance P-containing vesicles in a sucrose equilibrium gradient. Fractions containing substance P from FIG. 1 were pooled, concentrated, and then loaded on an equilibrium density sucrose gradient (25-50%). Fractions were analyzed for A, substance P content (ON); B, substance P content (LGN/SC) by RIA; C, individual synaptic vesicle membrane proteins (SV2, synaptotagmin, and synaptophysin) (ON); D, individual synaptic vesicle membrane proteins (SV2, synaptotagmin, synaptotagmin, synaptophysin, and synaptobrevin) (LGN/SC); E, synaptotagmin IV (LGN/SC) by Western blot.

FIGS. 3A-3E disclose co-sedimentation of vesicle-associated proteins with substance P inLGN/SC microsomes fractionated by size and density. Fractions containing substance P from FIG. 2 were analyzed for A, secretogranin II; B, βAPP (C8); C, Rab3, D, α-synuclein; E, BDNF by Western blot.

FIG. 4 discloses immunoadsorption of synaptic vesicle membrane proteins from substance P-containing fractions. Fractions containing substance P from FIG. 2 were immunoadsorbed with mouse IgG or synaptophysin Ab linked magnetic beads (2 or 4 mgs) (Dynal) as per the manufacturers instructions. Samples were analyzed for specific synaptic vesicle membrane proteins (SV2, synaptotagmin, synaptophysin, and synaptobrevin) by Western blot.

FIGS. 5A and 5B disclose electron micrographs of an immunolabeled, negatively stained DCV preparation from LGN/SC. Fractions containing substance P from an equilibrium gradient were fixed, adhered to formvar, carbon coated nickel grids and A, immunolabeled with mouse IgG and colloidal gold conjugated antibodies (12 nm); or B, immunolabeled with synaptophysin and colloidal gold conjugated antibodies (12 nm).

FIGS. 6A-6D disclose electron micrographs of immunolabeled thin sections from a DCV preparation from LGN/SC. Fractions containing substance P from an equilibrium gradient were fixed, embedded, sectioned and immunolabeled with A, rabbit IgG an colloidal gold conjugated synaptotagmin and colloidal gold conjugated antibodies (12 nm); and D, BDNF and colloidal gold conjugated antibodies (6 nm).

### DETAILED DESCRIPTION

In neurons, neuropeptides and other synaptic components are transported in an anterograde manner down the axon in vesicles to the synapse using molecular motors, or the kinesin family. In the synapse, these neuropeptides are found in dense core vesicles (DCVs), and following calcium-mediated exocytosis, they interact with receptors on the target cell. The inventors have developed a rapid, large-scale technique for purifying peptide-containing DCVs from specific nuclei in the central nervous system. The invention is based on experiments using differential velocity gradient and equilibrium gradient centrifugation, in which neuropeptide-containing DCVs were separated by size and density from optic nerve (ON) and its termini, the lateral geniculate nuclei and the superior colliculi. Isolated DCVs contain neuropeptides (substance P), brain-derived neurotrophic factor, synaptic vesicle (SV) membrane proteins (SV2, synaptotagmins, synaptophysin, Ra3 and synaptobrevin), SV-associated proteins (α-synuclein), secretory markers for DCVs previously isolated (secretogranin II), and β-amyloid precursor protein. Using electron microscopic techniques, DCV were also visualized and shown to be immunoreactive for neuropeptides, neurotrophins, and SV membrane proteins. Because of the interesting group of physiological and potentially pathophysiological proteins associated with these vesicles, this isolation procedure, applicable to other CNS nuclei, represents an important research tool for identifying bioactive compounds and biological targets.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

General Definitions. The term “neurotransmitter” refers to a chemical substance which delivers nerve impulses across a synapse between certain types of nerve cells. Upon a nerve impulse, a neurotransmitter is released at the end of a nerve axon. The neurotransmitter may diffuse across a synapse and is capable of creating subsequent nerve impulses. While in the nerve, the neurotransmitter is stored in synaptic vesicles.

The term “neuropeptide” refers to polypeptides which also act as neurotransmitters. Neuropeptides are distinguished from the common neurotransmitters by the neuroendocrine activity that results from their release by synaptic vesicles. As used herein, the neuropeptide is the marker, or protein of interest, within the vesicle chosen for purification.

The term “synaptic vesicle” refers to vesicles within the cytoplasm of the presynaptic nerve ending of neurons. These vesicles encompass neuropeptides and various neurotransmitters which are released into the synapse by a fusion of the synaptic vesicle with the presynaptic membrane. “Classic synaptic vesicles” and “dense core vesicles” are subtypes of the synaptic vesicle. The classic synaptic vesicles contain neurotransmitters. Dense core vesicles contain neuropeptides, and in some cases catecholamines as well as biogenic amines. The dense core vesicle refers to the type of synaptic vesicle isolated for purification by the present invention.

As used herein, the term “isolated” means that the referenced material is removed from the environment in which it is normally found. Thus, an isolated biological material can be free of cellular components, i.e., components of the cells in which the material is found or produced. In the case of an isolated protein, the protein may be associated with other proteins or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. Purity can be evaluated by chromatography, gel electrophoresis, immunoassay, composition analysis, biological assay, and other methods known in the art.

Methods for purification are well-known in the art. For example, nucleic acids can be purified by precipitation, ultracentrifugation and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

The term “large scale” as used herein refers to the quantity of protein marker obtained. Preferably, large scale production is greater than 20-fold; more preferably 100-fold; and more preferably at least 1,000-fold. Most preferably, large scale would result in 1,750-fold production of protein.

The term “protein of interest” refers to a protein the detection of which is desired and that is contained within the dense core vesicle.

### Dense Core Vesicles

In the present invention, the dense core vesicles (DCVs) containing the peptide marker are separated through homogenization and centrifugation of neuron particles from brain tissue. After a low speed centrifugation to separate out large cell debris, the DCVs are separated as a microsome preparation. The preparation is centrifuged again to produce a 7-fold increase. The DCVs, as a part of the preparation, are resuspended in hypotonic buffer and are layered on a sucrose velocity gradient which will separate the DCVs by size. The DCVs which deposit between 10-50% are collected and the DCV content is measured. The DCV fractions containing the highest ratios of peptide are pooled and centrifuged onto a 60% sucrose pad to allow a concentrated collection without breaking the DCVs. The DCVs are then layered onto a continuous sucrose gradient of 25-50% sucrose and are centrifuged to equilibrium, thereby separating the DCVs by density. The purified DCV fractions are then collected and are ready for analysis.

### Purification of the Dense Core Vesicles

The present invention advantageously provides a method of purifying peptide-containing vesicles. The inventors have discovered that it is possible to localize specific markers, or peptides, of interest and increase their production for use in various research protocols. Therefore, the first step is to identify a collection of proteins that individually show significant association with a particular disease.

Any sample that is likely to contain a protein of interest may be tested. Such samples include nerve and their termini dissected from the appropriate central nervous system region. In one embodiment, the nerves from the hypothalamus may be isolated to study proteins involved in eating disorders. One embodiment may focus on nerves from the temporal lobe, basal forebrain or hippocampus to study their role in Alzheimer's disease. In another embodiment, the nerves from the striatum could be investigated for proteins involved in Parkinson's Disease and Huntington's disease. In yet another embodiment, the ventral tegmentum could be studied for proteins involved in schizophrenia. In other embodiments, the motor cortex could be studied for amyotrophic lateral sclerosis or the lateral geniculate nuclei and superior colliculi could be studied for glaucoma. The possibilities can potentially carry over to every brain region.

Homogenization of the dissected sample is necessary to separate the tissue while preserving the cellular components. In a preferred embodiment, 8 strokes of a motor driven Teflon dounce homogenizer is followed by 8 strokes of a glass dounce homogenizer. However, another embodiment may use either Teflon or glass alone and varying numbers of strokes. The various methods may be used so long as necessary cellular components remain intact.

Various buffers are necessary throughout the preparation of the purified vesicles. The homogenization process is carried out in an appropriate homogenization buffer that would allow tissue particles to remain intact. In the preferred embodiment, the homogenization buffer would consist of an adequate amount of 320 mM sucrose, 1 mM TEA pH 7.2, 30 μg/ml phenylmethyl-sulfonyl fluoride, 0.25 μg/ml aprotinin, and 0.5 μg/ml leupeptin (protease inhibitors). Another embodiment may use an alternative buffer, perhaps with different protease inhibitors, assuming they are appropriate for homogenization purposes. During the resuspension after centrifugation, the pellet is placed in a hypotonic buffer. In a preferred embodiment, the buffer would consist of 40 mM sucrose, 1 mM TEA pH 7.2 and protease inhibitors. In one embodiment, another buffer may be used, possibly with variant inhibitors, but preferably a hypotonic buffer that would allow larger cell organelles to break open, but keep dense core vesicles intact.

Centrifugation is a necessary step to separate nuclei, unbroken cells, and large debris. In a preferred embodiment, the homogenate is pelleted at a low speed of 1200×g. However, in an alternative embodiment, various low speed centrifugation may be administered to remove the large particles. In one embodiment, another technique, such as filtering, could be used to achieve the same effect of separating out the larger particles. A subsequent centrifugation of the supernatant would allow the necessary microsomes to be separated for further purification. A preferred embodiment would centrifuge the supernatant at 100,000×g for 2 hours. This would yield a 7-fold increase in microsomes in comparison to another embodiment where the supernatant could be subjected to 10,000-20,000×g for 20 minutes to obtain a synaptosome. However, the product yield in the latter procedure would not be maximized. In another embodiment, a sizing column may be used to separate the tissue components, however with lower yield.

Sucrose gradients provide the means to separate the cell fractions first, by size, followed by density to substantially increase the product yield. The initial velocity gradient allows the purification by size; a subsequent equilibrium gradient allows the purification by density. In a preferred embodiment, the percentage sucrose on the velocity gradient containing DCVs is 29-42% while the percentage sucrose on the equilibrium gradient containing DCVs is 38.5-45.5%. Once the fractions have been purified by size, those fractions with high levels of protein are pooled and centrifuged. In the preferred embodiment, the fractions are centrifuged at 100,000×g for 2 hours onto a 60% sucrose pad. In another embodiment, it would be possible to pellet the fractions, however, the sucrose pad has enabled concentration of the fractions without breaking them, yielding a higher level of product. Once the subsequent equilibrium density is completed, the fractions are centrifuged to equilibrium. In a preferred embodiment, the centrifugation is done for 18 hours at 92,000×g.

### Analyzing the Dense Core Vesicles

DCVs purified in accordance with the invention provide a rich source of biological material, much of which may be new or poorly characterized because, absent purification of DCVs to the degree made possible by the invention, they were in such low abundance they were essentially undetectable. This material includes biologically active compounds, which may form the basis of therapeutics or may be lead compounds for developing a therapeutic. Such material can also be a new target for drug screening. In addition, the presence or amount of one or more compounds, or a particular “fingerprint” of a group of compounds, provides a powerful analytical tool for evaluating neuronal status, i. e., the health of the neuron.

Analysis of the DCVs can be carried out by using an array of methodologies including, but not limited to, radioimmunoassay, Western blot analysis, immunoadsorbtion techniques, and fixed vesicle preparations with immunolabeling.

Characterization of the complement of expressed protein mixtures can be done using two-dimensional (2D) polyacrylamide gel electrophoresis, developed in late 1960s. This technique resolves complex protein mixtures first by isoelectric focusing using carrier ampholytes and/or immobilised pH gradients, followed by separation according to size using traditional polyacrylamide gel electrophoresis under denaturing conditions. Separated proteins can be identified by their unique position on the obtained 2D gels and by their quantity using quantitative gel imaging systems.

Protein identification can be further clarified using mass spectrometry (MS) techniques. In using MS techniques, the 2D gel-separated proteins are first excised and digested (typically with trypsin). Then, the peptides obtained are typically identified using matrix-assisted laser desorption ionization-time of flight (MALDI/TOF) MS technique, followed by database mass matching. Further confirmation of peptide identification can be obtained using tandem mass (fragmentation) spectrometry (MS/MS) techniques with collision-induced dissociation (CID) to fragment the peptide enabling an amino acid sequence to be generated. See, e.g., International patent applications WO98/23950 and WO99/63351, as well as in U.S. Pat. No. 6,064,754. Pre-fractionation of the complex protein mixtures prior to the 2D gel separation improves the traditional technique and allows for lower abundace proteins to be separated and identified.

One skilled in the art can identify sequence information from peptides analyzed by mass spectrometry and/or tandem mass spectrometry using various spectral interpretation methods and database searching tools. Examples of some of these methods and tools can be found at the Swiss Institute of Bioinformatics web site on the World Wide Web at www.expasy.com, and the European Molecular Biology Laboratory web site on the World Wide Web at www.mann.embl?heidelberg.de/Services/PeptideSearch/.

More recently, one or both dimensions of electrophoretic separation have been substituted with chromatography (Davies et al., Biotechniques 1999, 6:1258-61; Senior, Mol. Med. Today 1999, 5:326-327; Gygi et al, Nature Biotechnology 1999, 17: 994-999; Wall et al, Anal. Chem. 2000, 72: 1099-111). Chromatography provides an alternative approach, using the same basic principle of protein separation in more than one dimension, followed by protein identification using MS.

More recently, another completely different technology has been applied for proteomics research, which is readily adapted to analyzing the contents of the DCVs. This technology employs arrays of affinity ligands (antibodies or other agents) immobilised on a variety of solid supports (Soloviev, Drug Discov. Today 2001, 6(15):775-777; Arenkov et al, Anal. Biochem. 2000, 278(2):123-31;Vasiliskov et al, Biotechniques 1999, 27(3):592-4, 596-8; Zlatanova et al, Methods Mol Biol. 2001, 170:17-38; Zhu et al, Nat Genet 2000, 26(3):283-9; Haab et al, Genome Biol. 2001, 2(2):RESEARCH0004; MacBeath and Schreiber, Science 2000, 289:1760-1763; Huang et al, Anal Biochem. 2001, 294(1):55-62). Using arrayed affinity ligands avoids the need for protein separation, as all of the spotted reagents are spatially separated and their positions known. The use of fluorescently labeled protein mixtures further simplifies protein detection and quantitation. Further increases in protein array sensitivity and signal-to-noise ratio were reported using time resolved fluorescence (Luo and Diamandis, Luminescence 2000, 15(6):409-13) and planar waveguides as protein immobilisation substrates (Weinberger et al, Pharmacogenomics 2000,1(4):395-416; Pawlak et al, Faraday Discuss. 1998, 111:273-88).

In another embodiment, peptide immunoassays may be used to analyze the quantitative peptide content of the purified DCVs, wherein the purified DCVs are extracted with acetic acid and insoluble debris is removed by centrifugation. The supernatant is collected and neutralized with NaOH. Radiolabeled peptide and antibody (1:80,000 dilution) recognizing mature peptide are incubated with samples and standards ranging from 1.56 to 400 fmol. at 4 degrees for 18-48 hours. A preferred embodiment would use 2N acetic acid, ION NaOH, and [¹²⁵I] radioisotope. Unbound radiolabeled peptide is extracted with Dextran (40 kD)-coated charcoal. Counts are determined in a Packard gamma counter and values calculated from a logarithmic standard curve (Floor et al., Neuroscience Lett (1982), 60: 231-7).

In another embodiment, Western blot analysis can be used to identify the specific proteins within the DCVs. Using the purified DCVs, proteins are separated using SDS-PAGE with polyacrylamide gels. A preferred embodiment would use either tris-glycine or tris-tricine. Gels are transferred to PVDF membranes and blocked with 5% mild in PBS with 0.1% Tween. Membranes are then probed with primary antibodies at various dilutions, washed, and probed with a secondary antibody. The specific protein bands are detected using enhanced chemiluminescent reagents (Thoidis, G. et al., J. Biol. Chem (1999), 274: 14062-6).

In another embodiment, specific peptide and protein content of DCVs can be analyzed through an immunoadsorbtion of vesicles. The purified DCVs are immunoadsorbed using M-500 subcellular Dynabeads, according to manufacturer's instructions. Beads are prepared with select antibodies attached to them. One embodiment may have synaptophysin, synaptotagmin, or mouse non-immune IgG antibodies. Approximately 200 ug of protein from purified DCVs are incubated with Dynabeads, PBS, and 5% fetal bovine serum. A preferred embodiment would contain 2-4 mg Dynabeads, incubated for 1-12 hours at 4° C. with constant agitation. The beads are resuspended in 2X Laemmli buffer. The specific peptide and protein content can then be determined with SDS-PAGE, Western blot analysis, or radioimmunoassay.

Another embodiment can use fixed DCV preparations for immunolabeling to analyzing peptide content. Purified DCVs with the highest protein content are fixed and negatively stained (Thoidis et al., J. Biol. Chem (1999), 274: 12062-6). A preferred embodiment would use purified vesicles with at least 200 ug/ml protein concentration fixed with 2% paraformaldehyde in PBS for 10 minutes. The DCVs are adhered to 200 mesh carbon and formvar-coated grids for negative staining. The grids are then blocked with 2% gelatin, washed with 0.02M glycine/PBS and 0.1% BSA/PBS, and incubated with appropriate antibody. A preferred embodiment would incubate with primary antibody diluted in 1% BSA/PBS for 30 minutes followed by washes of 0.1% BSA/PBS. The antibody/antigen complex is stabilized with glutaraldehyde in PBS, washed and stained. A preferred embodiment would stain with 4% uranyl acetate. Electron micrographs are then obtained using a Philips EM300 electron microscope.

Another embodiment would use purified DCVs and fix them with 4% paraformaldehyde; 1% lysine, 2% sodium periodate in 0.1 M phosphate buffer (pH 7.2) for 2-3 hours. The fixed vesicles are pelleted at 14,00×g using a tabletop microfuge, washed with PBS, and fixed with 2% osmium tetroxide. The pellets are rinsed in PBS, dehydrated in ethanol followed by propylene oxide and embedded in Embed 8120. Blocks are sectioned on RMC MT7000 Ultramicrotome and thin sections were mounted on uncoated 200-400 mesh nickel grids. Electron micrographs are obtained with a Philips EM 300 electron microscope. Grids with sections are blocked with 1% BSA in 0.1 M tris buffer, pH 7.4., for 15 minutes and incubated with antibody for 0.5 to 2 hours at room temperature. Grids are washed 3 times with 0.05M Tris buffer, pH 7.4, followed by 3 times with 0.05M Tris buffer, PH 7.4, and 1% BSA. Sections are incubated in colloidal gold-conjugated antibody in 0.1M tris buffer, pH 8.3 and 1% BSA for 30 minutes at room temperature followed by three washes of all buffers. Grids are stained with 3% uranyl acetate and dried. Electron micrographs are then obtained.

### Uses and Benefits

Use in Clinical Studies. The diagnostic methods of the present invention can assist in an array of uses, e.g., clinical screening, diagnosis, prognosis, therapy and prophylaxis, as well as for drug screening and drug development. In one embodiment, the invention can assist in monitoring a clinical study, e.g. to evaluate peptide containing vesicles for disease therapeutics.

In another embodiment, the methods and compositions of the present invention can be used in animal models, for example to study fetal malnutrition. In another embodiment, the invention can be useful in human models through biopsy, for example, when the hippocampus is removed for the prevention of seizures. Furthermore, another embodiment can use the present invention to assist in determining the relationship of protein levels with disease pathology.

### EXAMPLES

The following Example(s) illustrate the invention, but are not limiting.

### Example 1

### Isolation and Characterization of Substance P-Containing Dense Core Vesicles from Rabbit Optic Nerve and Termini.

Example 1 has selected the rabbit optic nerve (ON) and the nuclei in which it terminates to study neuropeptides and DCVs. Rabbits use neuropeptides, such as neurokinins, as secondary neurotransmitters. Neurokinins, which include substances P and K, are present in over 50% of rabbit RGCs (Brecha et al., Nature 1987, 327: 155-8). The Example describes here a method for isolating DCVs from the ON and its terminal nuclei, the lateral geniculate nucleus (LGN) and superior colliculus (SC), which can be utilized to isolate large quantities of DCVs from other nuclei in the CNS. The Example characterizes the resulting vesicles in terms of the morphology and protein constituents. The findings are published in the Journal of Neuroscience Research 2000, 62: 830-9.

### Materials and Methods

Materials. Reagents were purchased from Sigma Chemicals unless otherwise stated. 35S-Protein labeling mix and 125I-Bolton-Hunter reagents were purchased from Dupont NEN. Whole rabbit brains with ON attached were purchased from Pel-freez Biologicals. BCA protein assay kit and Super enhanced chemiluminescent (ESL) reagent was purchase from Pierce. Enhanced Chemiluminescent (ECL) reagent and Hyperfilm MP were purchased from Amersham and/or Pierce. M-500 Subcellular Dynabeads were purchased from Dynal. Rabbit anti-BDNF antibody was purchased from Research Diagnostics Incorporated. Rabbit anti-secretogranin II antibody was purchased from Biodesign International. Mouse anti-Rab 5 antibody was purchased from Transduction Laboratories. Mouse anti-bAPP (6E10) antibody was purchased from Serotec. Mouse anti-SV2 and mouse anti-synaptotagmin antibodies were a generous gift from Dr. Kathy Buckley, Harvard Medical School. Mouse anti-synaptophysin, mouse anti-synaptobrevin, and mouse anti-Rab3A antibodies were a generous gift from Dr. Reinhart Jahn, Max Planck Institute. Mouse anti-synaptotagmin IV antibody was a generous gift from Dr. Harvey Herschman, UCLA Medical School. Rabbit anti-bAPP (C8) antibody was a generous gift from Dr. Dennis Selkoe, Harvard Medical School and Brigham and Women's Hospital. Mouse anti-a-synuclein antibody was purchased from Zymed Laboratories Inc. Goat anti-rabbit-HRP and goat anti-mouse-HRP antibodies were purchased from Rockland Immunochemicals. Colloidal gold-conjugated goat anti-mouse and anti-rabbit antibodies were purchased from Jackson Laboratories. Electrophoretic gels, PVDF membrane, and molecular weight markers were purchased from Biorad and/or Novex. Supplies for electron microscopy were obtained from Electron Microscopy Sciences.

Preparation of Peptide-containing Vesiclesfrom Rabbit Visual System. Microsomes were prepared from rabbit ON, LGN, and SC. Briefly, nerve and nuclei were dissected from 10 young New Zealand rabbit brains. Tissue was diced and then homogenized using a motor driven Teflon dounce homogenizer (8 strokes) followed by a glass dounce homogenizer (8 strokes) in 40 mls of homogenization buffer (320 mM sucrose, 1 mM TEA pH 7.2, 30 mg/ml phenylmethyl-sulfonyl fluoride, 0.25 mg/ml anti-pain, 0.25 mg/ml aprotinin, and 0.5 mg/ml leupeptin [protease inhibitors]). Nuclei, unbroken cells, and large cell debris was then removed using low-speed centrifugation (1200×g). Microsomes were pelleted by centrifuging the supernatant at 100,000×g for 2 h. Pellets were resuspended in resuspension buffer (40 mM sucrose, 1 mM TEA pH 7.2 and protease inhibitors). Continuous gradients were formed using a Gibco BRL gradient maker and gradient linearity was confirmed by refractive index (data not shown). The resuspended pellet was then layered on a continuous gradient of 10-50% sucrose (w/v) in 1 mM TEA pH 7.2 and protease inhibitors and centrifuged at 86,000×g for 2 h. Fractions were collected and protein concentration and substance P concentration was measured. Fractions which contained high ratios of substance P to protein were pooled and centrifuged (100,000×g for 2 h) onto a 60% sucrose pad. Material on the pad was collected, diluted 1:1 with resuspension buffer, layered on a continuous sucrose gradient of 25-50% sucrose (w/v), and centrifuged to equilibrium (18 h at 92,000×g). Fractions were collected and protein and substance P concentrations were measured.

Substance P Radioimmunoassay. Substance P was measured using a radioimmunoassay previously described (Floor et al., Neuroscience 1982, 7: 1647-55). Briefly, samples were extracted with 2N acetic acid and insoluble debris was removed by centrifugation. The supernatant was collected and neutralized with 10N NaOH. Radiolabeled (1251) substance P and rabbit anti-substance P (1:80,000 dilution), recognizing only the mature form of substance P, were incubated with samples and standards (1.56-400 fmols) at 4° C. for 18-48 h. Unbound radiolabeled substance P was extracted from each sample using Dextran (40 kD)-coated charcoal. Counts were determined in a Packard gamma counter and values were calculated from a logarithmic standard curve.

Western Blot Analysis. DCVs were prepared as described above. To concentrate fractionated ON microsomes, fractions were pooled in groups of four (1-4, 5-8, 9-12, etc.) and proteins were precipitated using 10% trichloro-acetic acid (TCA). Precipitated proteins were pelleted at 14,000×g for 15 minutes and resuspended in phosphate buffered saline (pH 7.2). Proteins were separated using SDS-PAGE with either tris-glycine (Laemmli, Nature 1970, 227: 680-5) or tris-tricine (Schagger and von Jagow, Anal Biochem. 1987, 166: 368-79) polyacrylamide gels. Gels were transferred to PVDF membranes and blocked with 5% milk in PBS with 0.1% Tween (PBS-T). Membranes were probed with primary antibodies at a variety of dilutions, washed, and probed with goat anti-mouse IgG-HRP (1:10,000 dilution) or goat anti-rabbit IgG-HRP (1:20,000 dilution) as a secondary antibody. Specific protein bands were detected using enhanced chemiluminescent reagents.

Immunoabsorbtion of Vesicles. DCVs were prepared as described above. Vesicles were immunoabsorbed using M-500 subcellular Dynabeads according to the manufacturer's instructions. Briefly, beads were prepared which had synaptophysin, synaptotagmin, or mouse non-immune IgG antibodies attached to them. Approximately 200 mg total protein from peptide-containing fraction were incubated with 2 to 4 mg Dynabeads, PBS, and 5% fetal bovine serum for 1-12 h. at 4° C. with constant agitation. Beads were then resuspended in 2X Laemmli sample buffer. Specific peptide and protein content was determined using SDS-PAGE, Western blot analysis, and radioimmunoassay.

Fixed Vesicle preparationfor Immunolabeling. DCVs were prepared as described above. Substance P-containing fractions were fixed and negatively stained as previously described (Thoidis et al., J Biol Chem. 1999, 274: 14,062-6). Briefly, vesicles from fractions which contained the highest amount of substance P and a protein concentration of at least 200 mg/ml were fixed with 2% paraformaldehyde in PBS for 10 minutes and adhered to 200 mesh carbon and formvar-coated grids for negative staining. Grids were blocked with 2% gelatin and then washed with 0.02M glycine/PBS and 0.1% BSA/PBS. Grids were incubated with primary antibody diluted in 1% BSA/PBS for 30 min (mouse non-immune serum, 1:50; mouse anti-synaptophysin, 1:50), followed by 4 washes in 0.1% BSA/PBS. Grids were then incubated for 30 min with goat anti-mouse colloidal-gold antibody (12 nm) diluted in 1% BSA/PBS (1:10) and washed in 0.1% BSA/PBS. The antibody/antigen complex was stabilized with 1% glutaraldehyde in PBS, washed (PBS followed by H2O), and stained with 4% uranyl acetate. Electron micrographs were obtained using a Philips EM300 electron microscope.

DCVs were prepared as described above. Vesicles from fractions which contained the highest amount of substance P and protein were fixed with 4% paraformaldehyde; 1% lysine; 2% sodium periodate in 0.1 M phosphate buffer, pH 7.2 for 2-3 h. Fixed vesicles were pelleted at 14,000×g in a tabletop microfuge. Pellets were washed with PBS and fixed with 2% osmium tetroxide. The pellets were then rinsed in PBS, dehydrated in ethanol followed by propylene oxide, and embedded in EMbed 8120. Blocks were sectioned on a RMC MT7000 Ultramicrotome and thin sections were mounted on uncoated 200-400 mesh nickel grids. Electron micrographs were then obtained using a Philips EM 300 electron microscope.

Grids containing sections were blocked with 1% BSA in 0.1 M tris buffer, pH 7.4 (Buffer A) for 15 min and then incubated with primary antibody (diluted in Buffer A; rabbit non-immune serum, 1:50; mouse anti-synaptotagmin, 1:50; rabbit anti-substance P, 1:100; rabbit anti-BDNF, 1:50) for 0.5-2 h at room temperature. The grids were then washed 3 times with 0.05 M Tris buffer, pH 7.4 (Buffer B), followed by 3 times with 0.05 M tris buffer, pH 7.4, and 1% BSA (Buffer C). Sections were incubated in colloidal gold-conjugated goat anti-rabbit (6 nm) or anti-mouse (12 nm) IgG diluted in 0.1 M tris buffer, pH 8.3 and 1% BSA for 30 min at room temperature followed by 3 washes each in Buffers B, C, and distilled water. Grids were then stained with 3% uranyl acetate and dried. Electron micrographs were then obtained using a Philips EM 300 electron microscope.

### Results and Discussion

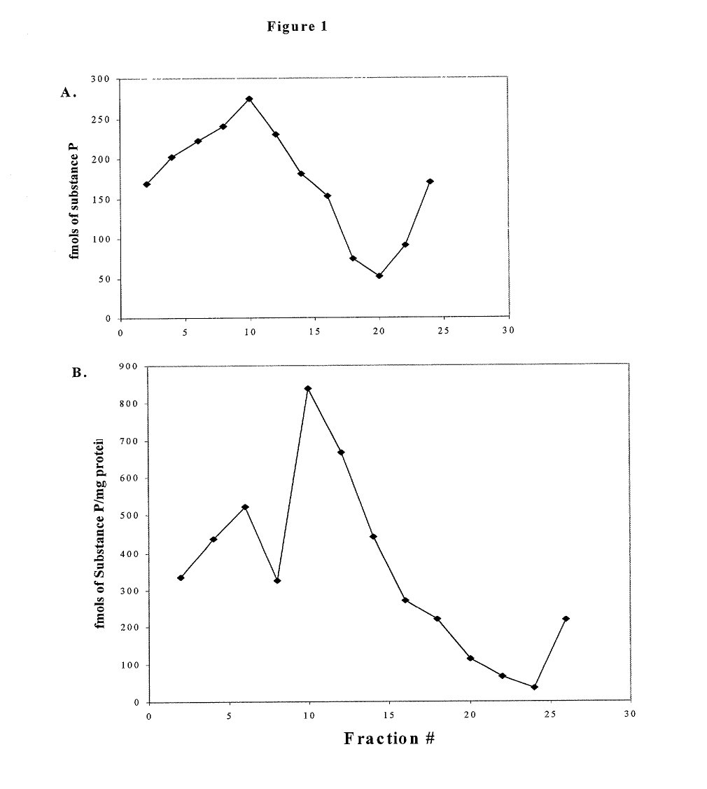

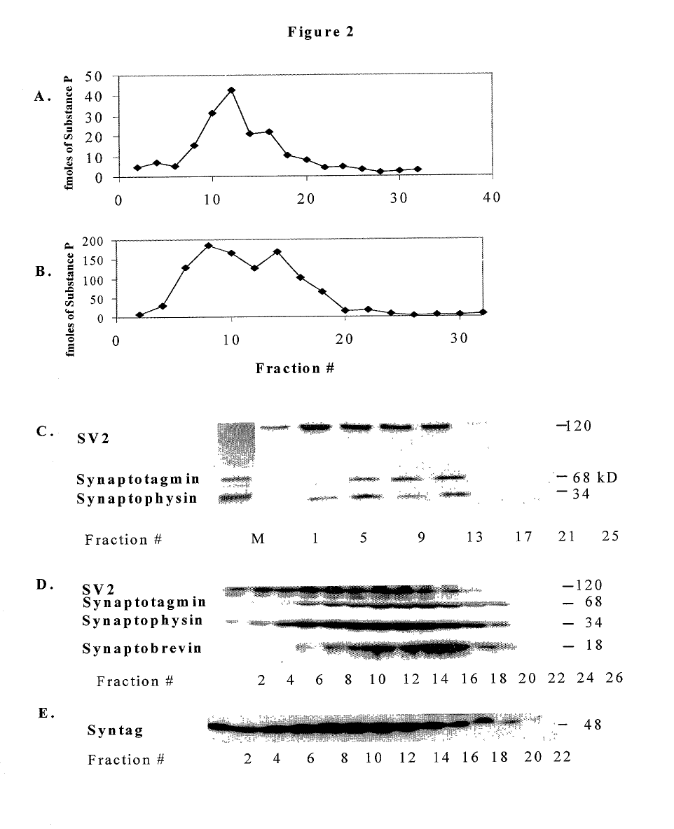

Isolation of DCVs. The protocol used to isolate substance P-containing DCVs combines separation of microsomes on the basis of size followed by separation by equilibrium density. This procedure achieves a 1,750-fold enrichment of substance P specific activity from the homogenate. FIG. 1 shows the distribution of substance P on the velocity (size) gradient. Since there is no appreciable difference in the substance P profile of ON (FIG. 1A) versus LGN/SC (FIG. 1B), the conclusion is that substance P-containing vesicles in transport (i.e. in the ON) are similar in size to those in the termini (LGN/SC). When substance P-containing material (fractions 3-16) was then fractionated by density, substance P is contained in fractions 6-18 with a consistent shoulder in fractions 14-18 in both ON and LGN/SC preparations (FIGS. 2A and B). This indicates that DCVs from both sources are similar.

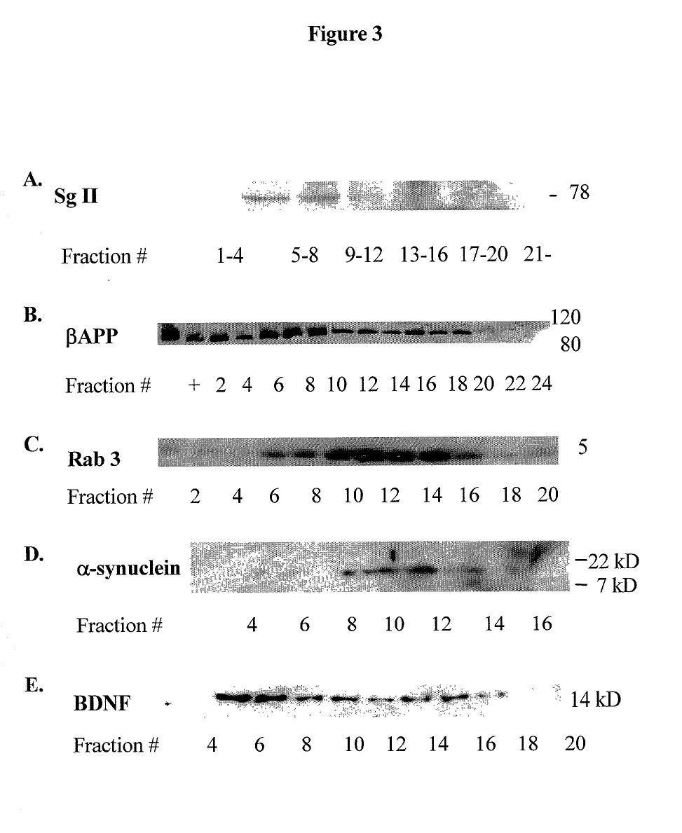

It should be noted here that classic SVs because of their small size and relative low density sediment much more slowly in the velocity gradient described here and also reach equilibrium at 30% sucrose corresponding to fractions 22-28. Therefore, they do not represent a significant contaminant. Western blot analysis of substance P-containing fractions detected co-sedimentation of substance P and several major protein markers of classic SVs, including SV2, synaptotagmin, synaptophysin, and synaptobrevin (FIGS. 2C and D), in DCVs from ON and LGN/SC. Additionally, another member of the synaptotagmin family, synaptotagmin IV, is also present (FIG. 2E) in DCVs from LGN/SC. The study has also detected co-sedimentation of substance P with the secretory marker secretogranin II, as well as with bAPP (FIGS. 3A and B). Neither of these proteins has been detected in significant amounts in classic SVs. Furthermore, cleavage products of bAPP (C99 and C83) are also detected (data not shown). Other vesicle-associated proteins are also detected including Rab3 (FIG. 3C), a-synuclein (FIG. 3D), and vacuolar H+-ATPase (data not shown). BDNF also colocalizes with substance P (FIG. 3E). The ER marker, calnexin, and an endosomal marker, the transferrin receptor, are depleted in fractions containing DCV membranes (data not shown). Additionally, Rab5 (a GTPase associated with endosome recycling) which is present in endosomal membranes does not localize with substance P (data not shown). These data appear to demonstrate the isolation of at least two different vesicle subtypes. While there is overlap for most analyzed proteins, the heavier vesicle contains more of the SV2, synaptotagmin IV, secretogranin II, bAPP, and BDNF while the lighter vesicle contains more of the synaptotagmin I, synaptobrevin, Rab 3, and a-synuclein. Both vesicle subtypes however, contain substance P and synaptophysin.

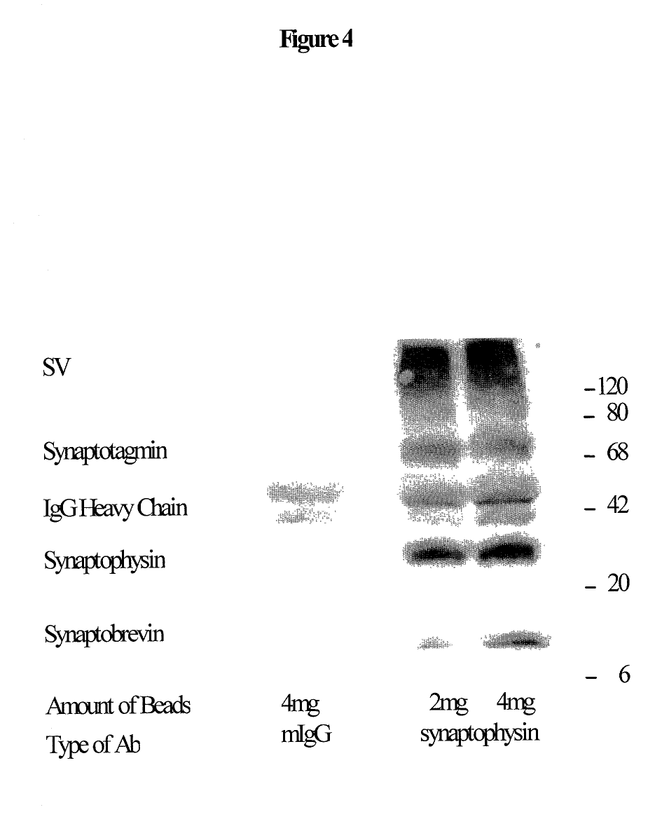

While the biochemical data obtained were consistent with the hypothesis that substance P containing fractions were DCVs, other experiments were performed to confirm this hypothesis. Western blot analysis of material immunoadsorbed on magnetic beads (Dynal) coated with synaptophysin (FIG. 4) visualized SV membrane proteins (SV2, synaptotagmin I, and synaptobrevin) co-precipitated with synaptophysin-containing vesicles. These proteins were not visualized in the control (mIgG) immunoadsorbtion. Furthermore, half (25 fmols) of the total (50 fmols) substance P is recovered by the anti-synaptophysin linked beads with control bead adsorbing considerably less (10 fmols).

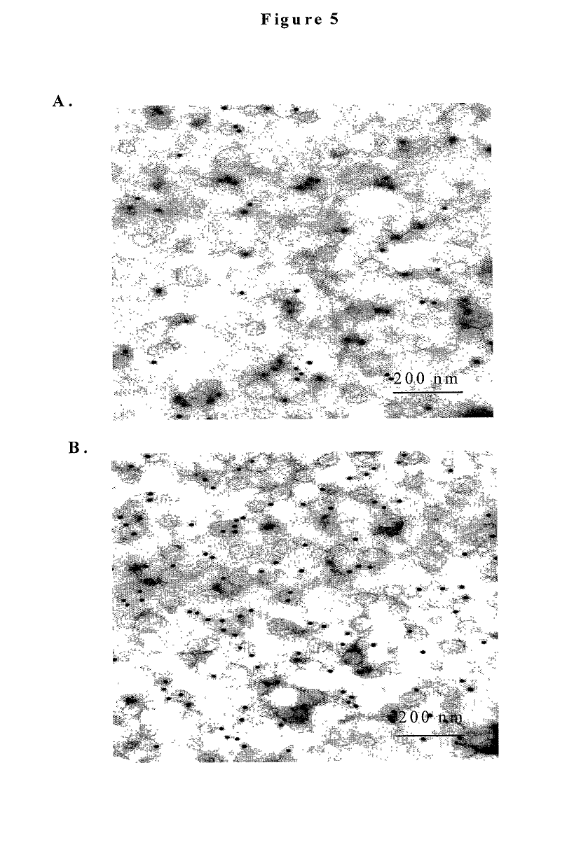

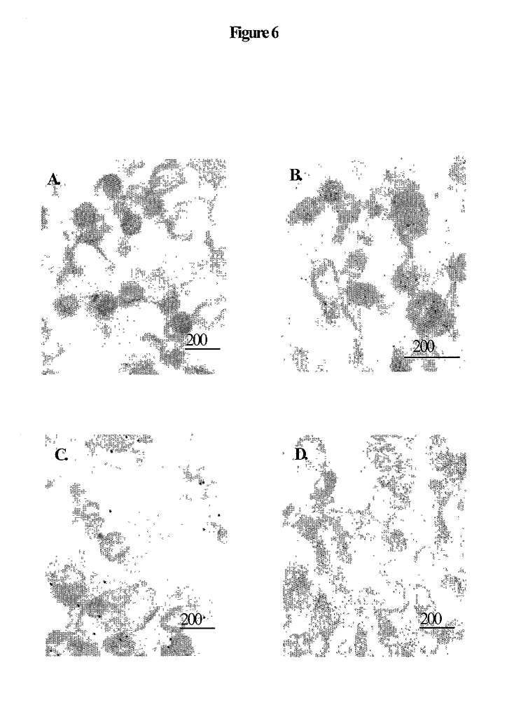

Visualization and immunolabeling of DCVs. Fixed peptide-containing vesicles were prepared, adhered to formvar carbon coated grids, negatively stained, and immunolabeled as described. FIG. 6 shows electron micrographs of these vesicles that have been immunolabeled with either non-immune mIgG (FIG. 5A) or mouse anti-synaptophysin (FIG. 5B) followed by colloidal gold conjugated goat anti-mouse IgG (12 nm diameter). These micrographs display a fairly homogeneous population of vesicles approximately 90±20 nm in diameter. There was significantly more (P<0.05) vesicle-associated immunolabeling with anti-synaptophysin compared to controls (mlgG) as determined by random sampling of micrographs and Student's t-test.

Fixed DCVs were also pelleted, embedded, sectioned, labeled with normal rabbit serum (6A), anti-substance P (6B), anti-synaptotagmin (6C), and anti-BDNF (6D) followed by colloidal gold-conjugated anti-rabbit or mouse IgG. These vesicle structures do not appear as homogeneous as those in FIG. 5 possibly due to changes resulting from the more rigorous fixation techniques (see Discussion). Additionally, vesicles appear larger (120±30 nm) in FIG. 6 as well. Vesicles labeled with rabbit serum (control) followed by colloidal gold show no immunolabeling (FIG. 6A), while vesicles labeled with rabbit anti-substance P, mouse anti-synaptotagmin, or rabbit anti-BDNF show significantly more immunolabeling (P<0.001). These data confirm those obtained in western blot analysis and immunoadsorption.

Discussion. Using a neuropeptide, substance P, as a marker, this study has isolated DCVs from the rabbit visual system. These vesicles sediment to a density of approximately 1.396±0.01 g/ml as determined by equilibrium density gradient fractionation and refractive index. A consistent finding in the substance P fraction profile was the presence of a shoulder in fractions 14-16 (FIG. 2). It is unclear what membrane pool this shoulder represents but two possibilities include DCVs from interneurons or other projection neurons in the LGN or SC, or DCVs from specific RGCs. Both possibilities are plausible especially when one considers that only half of RGCs make substance P. This peak could represent differences in peptide content from one RGC to another or a different source of substance P altogether. Further proof that the observed shoulder is physiologically relevant is demonstrated by the detection of specific protein co-sedimentation differences between the main substance P-containing peak and the shoulder. Further investigation of these possible subtypes is currently ongoing.

Using Western blot analysis, several proteins have been colocalized with substance P in fractionated ON and LGN/SC microsomes. These proteins include classic SV membrane proteins, (SV2, synaptotagmin I and IV, synaptophysin, and synaptobrevin; FIG. 3A and B), BDNF (FIG. 3E), the secretory marker, secretogranin II (FIG. 3A), bAPP (FIG. 3B), Rab3 (FIG. 3C) and a-synuclein (FIG. 3D). Furthermore, based on identification of specific protein markers, other organellar membranes known to be present in axons and synapses such as endosomes (transferrin receptor and Rab5) and endoplasmic reticulum (calnexin) are not significant contaminants. Therefore, this research demonstrates an excellent method for the isolation and purification of DCVs from the rabbit visual system in large quantity. It is also a reasonable assumption that this methodology can be applied to any nucleus in the CNS using a secretory marker like secretogranin II or a neuropeptide marker identified for a particular class of neurons characteristic of a specific nucleus.

Material from DCV-containing fractions, which have been fixed and adhered to formvar-coated grids and negatively stained, showed a fairly homogeneous population of vesicles approximately 90+/−30 nm in diameter (FIG. 5). DCVs fixed and embedded had more membrane irregularities and have diameters of approximately 120+/−30 nm (FIG. 6). These differences are likely due to the more rigorous fixation and dehydration techniques used in the embedding of DCVs. Both of these diameters are within reported variation of DCVs isolated in other systems (Floor et al., Neuroscience 1982, 7: 1647-55; Llona et al., Neurochem Int. 1994, 25: 573-81). Additionally, as evidenced by large membrane structures found only in embedded preparations (data not shown), these membranes are quite fusigenic making accurate observation of vesicle diameter more difficult in fixed preparations in which concentration by centrifugation is necessary. Through the use of immuno-EM, these vesicles are shown to contain specific proteins, such as classic synaptic vesicle membrane proteins (synaptophysin [FIG. 6B] and synaptotagmin [FIG. 7C]), substance P, and BDNF (FIGS. 7B and D).

Many, if not all, major membrane proteins of DCVs and SVs from whole brain or specific nuclei appear identical (Floor and Leeman, Neurosci Lett. 1985, 60: 231-7; Schmidle et al., Biochim Biophys Acta 1991, 1060: 251-6; Walch-Solimena et al., J Neurosci. 1993, 13: 3895-903; Winkler, Neurochem Res. 1997, 22: 921-32). Synaptophysin, which makes up approximately 1% of the total protein in classic synaptic vesicles (Navone et al., J Cell Biol 1986, 103: 2511-27) has been reported to be almost totally absent from DCVs in cell culture models of neurons, such as PC 12 cells or neuroendocrine cells such as vasopressin-containing magnocellular cells (Cutler and Cramer, J Cell Biol. 1990, 110: 721-30; Lah and Burry, J Neurochem 1993, 60: 503-12; Walch-Solimena et al., J Neurosci. 1993, 13: 3895-905). However, subsequent experiments have shown this protein to be present as a component of DCV membrane proteins from neurosecretory cells and neurons (Schilling and Gratzl, FEBS Lett. 1988, 233: 22-4). The difference may reflect differences between in vitro and in vivo systems. The rabbit RGC axons are 3 cm in length and it may be necessary for them to recycle DCV membrane proteins at the synapse. In contrast, cultured cells have no such lengthy transport problem being only 20 mm in diameter. Magnocellular cells have very few, if any, small synaptic vesicles which would make recycling the DCV membrane proteins at the synapse unnecessary. Certainly both recycling and retrograde transport of SV-associated proteins remain as possible trafficking scenarios.

DCVs from LGN/SC appeared to have significant quantities of synaptophysin as is demonstrated by Western blot, substance P recovery by immunoadsorbtion of synaptophysin-containing vesicles, and immuno-EM. Given that synaptophysin was also found in DCVs isolated from ON, it is likely that synaptophysin is part of the DCV biosynthesis and not simply added to DCVs in recycling or fusion events at the synapse. The seeming disparity between the amount of synaptophysin (i.e., relative amount of synaptophysin compared to synaptotagmin) in ON versus LGN/SC DCVs would suggest possibly both sources (cell body and synapse) of synaptophysin could contribute to DCV membrane proteins. The inability to detect Rab5 (a Rab associated with endosomal recycling) however, makes an hypothesis of DCV synaptophysin-association directly involving an endosomal compartment unlikely.

Two other DCV proteins, synaptotagmin I and IV, belong to the large synaptotagmin family of proteins. This family contains at least 12 members of which most have been localized in vesicle trafficking in organelles from many cell types (Sudhof and Rizo, Neuron 1996, 17: 379-88). Synaptotagmin I and IV have also been shown to be components of classic SVs and the sensitivity to Ca++, which triggers exocytosis, is altered by the ratio of the two family members present in the SV membrane (Littleton et al., Nature 1999, 400: 757-60). The data presented here suggest that a similar regulatory mechanism for exocytosis is used in DCVs and SVs.

The finding of a significant amount of BDNF in DCVs from both ON and LGN/SC may seem surprising in that there is evidence that during retinal development, BDNF is a target derived trophic factor for RGCs (Cohen-Cory et al., Dev Biol. 1996, 179: 102-15; Cohen-Cory and Fraser, Neuron 1994, 12: 747-61; Johnson et al., J Neurosci. 1986, 6: 3031-8; von Bartheld, Histol Histopathol. 1998, 13: 437-59). There is, however, recent data indicating that BDNF is synthesized and transported in an anterograde manner in the CNS and peripheral nervous system (Altar et al., Nature 1997, 389: 856-60; Zhou and Rush, Neuroscience 1996, 74: 945-53). It is possible to envision two functions for BDNF at the RGC synapse. One is that following release it is taken up by the same presynaptic terminals from which it is released and transported in a retrograde manner with its activated TrkB receptor to the RGC cell body, thereby functioning as an autocrine trophic factor. Secondly, BDNF can function to modify the postsynaptic cell following release. There are now several reports that BDNF can serve to either hyperpolarize or depolarize postsynaptic membranes (Chao, J Neurosci Res 2000, 59: 353-5; Jankowsky and Patterson, Mol Cell Neurosci 1999, 14: 273-86). These two postulated functions are not mutually exclusive. Further work is warranted in this system to delineate the function(s) of the DCV-associated BDNF.

Aggregation (in a manner similar to b-amyloid) of the protein, a-synuclein, has been postulated to be a causative agent in the formation of Lewy bodies (Narhi et al., J Biol Chem 1999, 274: 9843-6) and has been genetically linked to familial forms of Parkinson's disease with Lewy bodies (Polymeropoulos et al., Science 1997, 276: 2045-7). One of the proposed functions of a-synuclein is association with SVs through acidic phospholipids on the cytosolic surface with a potential role in synaptic plasticity (Clayton and George, J Neurosci Res. 1999, 58: 120-9; Davidson et al., J Biol Chem 1998, 273: 9443-9; Jenco et al., Biochemistry 1998, 37: 4901-9; Maroteaux et al., J Neurosci. 1988, 8: 2804-15). As is the case with other SV-associated proteins, a-synuclein also associates biochemically with DCVs. The antibody used (Zymed Laboratories Inc.) is reported to be specific for a-synuclein and does not crossreact with b- or g-synuclein (Baba et al., Am J Pathol. 1998, 152: 879-84). The physiological or pathological significance of this association remains to be established.

βAPP has been linked to Alzheimer's disease by genetic analyses from familial forms of AD and subsequent studies in animal models [reviewed in (Selkoe, Trends Cell Biol 1998, 8: 447-53)]. Previous studies in our lab demonstrate that bAPP is rapidly transported in a dense vesicle pool corresponding to DCVs (Amaratunga and Fine, J Biol Chem. 1995, 270: 17,268-72; Morin et al., J Neurochem 1993, 61: 464-73). The data presented here confirm that there is a significant association between bAPP and DCVs. Full length bAPP (FIG. 3B), sbAPP (data not shown) as well as bAPP cleavage products (data not shown) colocalize with DCVs from rabbit optic nerve and termini. These cleavage products include C99 and C83, raising the possibility that the DCV is an important compartment for bAPP processing. As with synaptotagmin IV, there is a significant amount of bAPP in membranes with slightly lower density than DCVs indicating the presence of another similar vesicle compartment. In fact previous work by our lab indicated the existence of a “light” transport vesicle containing bAPP but not synaptophysin (Morin et al., J Neurochem 1993, 61: 464-73).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A method of analyzing contents of purified dense core vesicles (DCVs), and which method comprises determining the contents of DCVs from extracted brain samples wherein the DCVs are purified at least 50-fold.

2. The method of claim 1, wherein the brain sample is rabbit optic nerve.

3. The method of claim 1, wherein the analysis is done by immunoassay.

4. The method of claim 1, wherein the analysis is done by Western blot.

5. The method of claim 1, wherein the analysis is done by chromatography.

6. The method of claim 1, wherein the analysis is done by mass spectroscopy.

7. The method of claim 1, wherein the analysis is done by immunoadsorbtion.

8. A method of purifying dense core vesicles, which method comprises
(a) centrifugating a resuspended pellet after homogenization of nerve and termini from dissected brain samples in order to obtain a microsome preparation;
(b) separating microsome preparation by a sucrose velocity size gradient;
(c) cetrifugating the microsome preparation onto a sucrose pad to yield a purified product; and
(d) collecting a quantity of the purified product from an equilibrium density gradient.

9. The method of claim 8, wherein the initial centrifugation of step (a) is low speed, followed by 100,000×g for 2 hours.

10. The method of claim 8, step (c) wherein the final quantity of the product is separated by density through centrifugation of 92,000×g for 18 hours.

11. The method of claim 8, wherein dense core vesicles were collected on the sucrose velocity size gradient at gradient levels between 29-42%.

12. The method of claim 8, wherein dense core vesicle were collected on the sucrose equilibrium density gradient at gradient levels between 38.5-45.5%.

13. A composition comprising purified dense core vesicles having nerve peptide or neurotransmitter.

14. A composition of claim 13, wherein the composition contains Substance P.

15. A composition of claim 13, wherein the purification is greater than 1,750-fold.
